(19) United States
(12) Patent Application Publication
(10) Pub. No.: US 2026/0272795 A1
(43) Pub. Date: Sep. 17, 2026

(54) STERIC-SUSPENSION-STABILIZED COSMETIC DISPERSION

(71) Applicant: CAPSUM, Marseille (FR)

(72) Inventors: Sébastien BARDON, VEYRIER DU LAC (FR); Mathieu GOUTAYER, SAINT MALO (FR)

(21) Appl. No.: 19/472,825

(22) PCT Filed: Apr. 12, 2024
(86) PCT No.: PCT/EP2024/059975

§ 371 Date: Oct. 6, 2025

(30) Foreign Application Priority Data
Apr. 13, 2023 (FR) FR2303691

(51) Int. Cl. A61K 8/11; A61K 8/20; A61K 8/34; A61K 8/37; A61K 8/46; A61K 8/49; A61K 8/67; A61K 8/73; A61K 8/81; A61K 8/85; A61K 8/92; A61Q 19/00

(52) U.S. Cl. CPC A61K 8/11; A61K 8/20; A61K 8/34; A61K 8/347; A61K 8/375; A61K 8/463; A61K 8/4973; A61K 8/678; A61K 8/73; A61K 8/733; A61K 8/8129; A61K 8/85; A61K 8/922; A61Q 19/00; A61K 2800/10; A61K 2800/43; A61K 2800/624; A61K 2800/654

(57) ABSTRACT

A composition, in particular a cosmetic composition, in the form of a dispersion comprising a dispersed phase in the form of capsules and a continuous aqueous phase, wherein: the capsules represent a volume fraction of between 50% and 70% relative to the total volume of the composition; the capsules comprise a shell and a core comprising at least one fatty phase comprising at least one active agent; the capsules comprise an Ri/Rc ratio of between 0.5 and 0.8, preferably between 0.6 and 0.8, and better still between 0.6 and 0.7, wherein: Ri corresponds to the radius of the core of the capsules; and Rc corresponds to the radius of the capsule.

The present invention relates to a composition in the form of a dispersion comprising a dispersed phase in the form of capsules and a continuous aqueous phase, wherein the capsules represent a volume fraction between 50% and 70% relative to the total volume of the composition, notably for a cosmetic application.

The preparation of compositions comprising at least one active agent often presents disadvantages, notably in terms of incompatibility of the active agent with other raw materials and/or its sensitivity to oxidation, or even its inability to be solubilized/dispersed in the phase comprising it.

Encapsulation presents itself as an ideal solution to overcome the aforementioned disadvantages. Indeed, encapsulation consists of encapsulating unstable active agents, thus allowing them to be isolated from other elements of the preparation, which preserves them from any risk of oxidation or contact with other incompatible raw materials. Encapsulation allows the active agents to be released only at the time of application on the surface to be treated, and thus guarantees an unaltered and high-quality active agent.

A first encapsulation solution consists of nano/micro-emulsions, notably described in U.S. Pat. No. 5,468,725 or WO2021156521, whose stability relies on very small-sized dispersed phase droplets and the use of surfactants. However, this technical solution is not entirely satisfactory, as nano/micro-emulsions have a milky appearance, and the presence of surfactants generally leads to a sticky feel. Moreover, surfactants are often irritating and have a poor image with consumers.

A second encapsulation solution consists of dispersions wherein the dispersed phase is in the form of macroscopic capsules dispersed in a continuous aqueous phase in the form of a suspensive aqueous gel thanks to the presence of hydrophilic gelling agents, chosen for example among carbomers or polysaccharides. Such dispersions, notably described in WO2010/063937, are generally transparent and have an attractive visual. However, it is known that some active agents present in the dispersed phase migrate to the continuous phase and lead to an opacification of the latter and/or interact with the hydrophilic gelling agents, leading to a drop in viscosity of this continuous aqueous phase. These degradations then give an opaque and/or non-suspensive finished product, potentially acidic and of inappropriate texture. Moreover, the use of a suspensive aqueous gel makes it difficult, if not impossible, to formulate the product as a spray. Finally, this type of dispersion requires the implementation of capsules with a sufficiently robust shell to ensure satisfactory kinetic stability. However, this robustness sometimes comes at the expense of the sensory quality since the application of such dispersions generally accompanies the presence of an undesirable residual membrane. It is this disadvantage that led the applicant to develop packaging equipped with a tangential filter to retain the shells, as described in WO2014/027039. Moreover, it is this disadvantage that also makes this type of dispersion incompatible with packaging equipped with a spray-type distribution pump.

The present invention aims to provide new compositions in the form of stable dispersions wherein the system ensuring the suspension of said capsules remains effective, including in the presence of high contents of active agent(s) in the dispersed phase.

Furthermore, the present invention aims to provide new compositions in the form of stable dispersions with an easy adjustment capacity in the rate of dispersed phase.

The present invention also aims to provide new compositions in the form of stable dispersions with an improved sensory quality.

Finally, the present invention aims to provide new compositions in the form of stable dispersions that are sprayable, i.e., offering a high aptitude for spraying.

Thus, the present invention relates to a composition, notably cosmetic, in the form of a dispersion comprising a dispersed phase in the form of capsules and a continuous aqueous phase, characterized in that:
the capsules represent a volume fraction between 40% and 80%, and in particular between 50% and 70%, relative to the total volume of the composition;
the capsules comprise a shell and a core comprising at least one fatty phase comprising at least one active agent; and
the capsules comprise a ratio Ri/Rc between 0.5 and 0.8, preferably between 0.6 and 0.8, and better still between 0.6 and 0.7, wherein:
Ri corresponds to the radius of the core of the capsules; and
Rc corresponds to the radius of the capsule.

Unexpectedly, the inventors observed that a composition according to the invention ensures a satisfactory suspension of the capsules, an easy adjustment in the content of the core of the capsules, and even a very good aptitude for spraying, including in the presence of high contents of active agent(s) in the dispersed phase.

Without wishing to be bound by any theory, the inventors believe that the minimum volume fraction required in macroscopic capsules allows the composition to be placed according to the invention in a regime close to close packing, which minimizes, or even makes negligible, the instabilities related to creaming or sedimentation phenomena, particularly creaming, of the capsules. One can therefore speak of “steric suspension” or “autonomous suspension,” each macroscopic capsule participating in the suspension of adjacent capsules.

This observation is all the more unexpected as a composition according to the invention, despite a high volume fraction in capsules, presents a very satisfactory sensory quality, notably resulting in the absence of residual shell (or “residual membrane”) or in the aptitude of the residual membranes, when present, to (i) not overly impact the visual of the composition applied to the material to be treated and (ii) to spread and disappear easily upon spreading.

This observation is even more unexpected as a composition according to the invention remains compatible with spraying, i.e., with a formulation in the form of a spray, despite a high volume fraction in capsules.

According to the invention, the pH of a composition is typically between 4.0 and 8.0, particularly between 5.0 and 7.0.

Unless otherwise indicated, in all that follows, it is considered that we are at room temperature (for example T=25° C.±2° C.) and atmospheric pressure (760 mm Hg, or 1,013·10⁵ Pa or 10¹³ mbar).

### Continuous Aqueous Phase

According to the invention, the continuous aqueous phase comprises at least water. In addition to distilled or deionized water, water suitable for the invention can also be natural spring water or floral water.

The continuous aqueous phase can serve as a dispersion medium for the capsules and ensure permanent hydration of their shell.

According to a particular embodiment, the continuous aqueous phase can comprise at least water and optionally at least one hydrophilic gelling agent, and thus be qualified as “aqueous gel.”

By “hydrophilic,” we mean to designate a gelling agent that is soluble or dispersible in water.

A hydrophilic gelling agent notably allows the fluidity of the continuous aqueous phase to be modulated, and thus the texture and/or sensory quality of the composition.

Preferably, the mass percentage of water in the continuous aqueous phase is at least 70%, notably from 70% to 98%, preferably from 80% to 95%, relative to the total mass of the continuous aqueous phase.

Preferably, the continuous aqueous phase represents a volume fraction less than 50%, preferably between 15% and 49%, and in particular between 30% and 40%, relative to the total volume of the composition.

As an additional hydrophilic gelling agent, one can cite:
natural gelling agents, notably chosen among algae extracts, plant exudates, seed extracts, microorganism exudates, such as alcasealan (INCI: Alcaligenes Polysaccharides), and other natural agents, particularly hyaluronic acid,
semi-synthetic gelling agents, notably chosen among cellulose derivatives and modified starches,
synthetic gelling agents, notably chosen among (meth)acrylic acid homopolymers or one of their esters, (meth)acrylic acid copolymers or one of their esters, AMPS (2-acrylamido-2-methylpropane sulfonic acid) copolymers, associative polymers, for example those described in FR2999921,
other gelling agents, notably chosen among clays, silicas such as those marketed under the names Aérosil® 90/130/150/200/300/380), and
their mixtures.

These hydrophilic gelling agents are described in more detail in FR3041251.

In particular, the hydrophilic gelling agents are preferably chosen from the group consisting of polyoxides, galactomannans, polysaccharides, glycosaminoglycans, polyols, and their mixtures.

Advantageously, the hydrophilic gelling agents are chosen from the group consisting of xanthan, carrageenan, locust bean, guar, gellan, hyaluronic acid, cellulosic derivatives, and their mixtures.

Of course, the person skilled in the art will ensure to choose the possible hydrophilic gelling agents and/or their quantity in such a way that the advantageous properties of a composition according to the invention are not or substantially not altered by the envisaged addition. These adjustments fall within the general knowledge of the person skilled in the art.

Preferably, a composition, in particular the continuous aqueous phase, does not comprise carbomer (or acrylic polymer).

Preferably, a composition, in particular the continuous aqueous phase, does not comprise a base, in particular alkali metal hydroxide, and notably NaOH (or soda).

Preferably, the continuous aqueous phase is liquid and is non-suspensive with respect to the capsules.

Preferably, the continuous aqueous phase has a high shear viscosity, as measured at 25° C. and under a shear stress of 100 s⁻¹, less than or equal to 100 mPa·s, preferably less than or equal to 50 mPa·s., in particular less than or equal to 25 mPa·s., and better still less than or equal to 10 mPa·s.

Preferably, a composition according to the invention is sprayable.

By “sprayable,” “spraying,” or “sprayable,” in the sense of the invention, we mean to designate the ability of a composition to be sprayed in the form of fine droplets by means of spray-type delivery devices (pumps), or even pressurization (aerosols).

Advantageously, the continuous aqueous phase has a yield stress value less than or equal to 1 Pa, in particular less than or equal to 0.1 Pa, especially less than or equal to 0.01 Pa, or even devoid of yield stress value. The yield stress value is measured at 25° C., using the following method.

The viscosity and yield stress value are measured using the following method:

All measurements are carried out with a DHR10 rheometer from Ta instrument equipped with a 40 mm diameter moving part forming a 1° cone—the measurement gap being 29 μm. The rheometer is controlled by the Trios software. Measurements are performed at 18° C., the temperature being controlled by a Peltier device. The rheological behavior is measured here using a shear sweep protocol (Flow sweep). Once the sample is in place and the temperature of 18° C. is reached, the sample is left to rest for 60 seconds so that it can relax its stresses. It is then subjected to a logarithmic shear sweep between 0.01 and 500 s⁻¹ with 5 points per decade. The viscosity (mPa·s) and stress (Pa) curves are plotted using the Trios software. The analysis of the stress curve by the software by applying the Herschel-Bulkley model allows the rheological behavior to be expressed according to the following equation:

τ=τ₀+k\dot{γ}ⁿ,siτ≥τ₀
where τ is the shear stress; τ₀ is the yield stress value; k is the consistency coefficient; \dot{γ} is the shear rate and n is the flow index.

Preferably, a composition according to the invention comprises less than 10%, preferably less than 5%, in particular less than 2.5%, or even is devoid of, alcohol(s), in particular ethanol, relative to the total weight of the composition.

By “alcohol,” we notably mean to designate a lower alkyl alcohol. A lower alkyl alcohol can be an aliphatic monoalcohol comprising from 2 to 6 carbon atoms. Such a lower alkyl alcohol can be ethanol.

The composition is preferably free of any lower alkyl alcohol.

In the sense of the present invention, “without ethanol” or “without alcohol” can also designate a cosmetic that is prepared by a preparation process not comprising preferably any step of adding a lower alkyl alcohol and no step of mixing an alkyl alcohol with other ingredients.

However, it cannot be excluded that traces of lower alkyl alcohol, notably traces of ethanol, may be present in some of the ingredients used in the preparation of the cosmetic according to the invention.

According to a particular embodiment, the continuous aqueous phase can further comprise at least one depolymerizing agent, notably when the shell of the capsules comprises at least one gelled polyelectrolyte, and in particular at least one polyelectrolyte reactive to multivalent ions, as described below.

In the context of the present description, “depolymerizing agent” means a compound capable of weakening the shell of the capsules to facilitate the application of the composition and in particular prevent the appearance of residual shell (or “residual membrane”) during application. A depolymerizing agent is notably described in WO2013/132082. According to one embodiment, the polyelectrolyte of the capsules is chosen among polyelectrolytes reactive to calcium ions, such as sodium alginate, and the depolymerizing agent is chosen among calcium chelating agents and salts capable of exchanging with calcium, such as EDTA.

Advantageously, the continuous aqueous phase is transparent so that the consumer can visualize the capsules.

### Capsules

A composition according to the invention comprises capsules that are macroscopic, and preferably monodisperse.

By “capsules,” we mean to designate, in the sense of the present invention, a macroscopic spherical element of the core/shell type, wherein the shell completely encapsulates the core and the core comprises at least one drop of fatty phase, the fatty phase comprising at least one active agent.

The capsules according to the invention form the “dispersed phase” or the “dispersed fatty phase.”

The shell can indifferently be designated by the terms “membrane” or “envelope.”

By “macroscopic,” “macroscopic capsule,” or “macroscopic dispersion,” we mean to designate, in the sense of the present invention, capsules visible to the naked eye, as opposed to microscopic capsules not visible to the naked eye. Thus, preferably, the capsules have a diameter (or size) greater than or equal to 250 μm, or even greater than or equal to 500 μm, particularly between 250 μm and 3,000 μm, better still between 500 μm and 2,000 μm, and especially between 750 μm and 1,000 μm.

### Shell

The shell completely encapsulating the core is advantageously a gelled envelope and/or an envelope deriving from a complex coacervation reaction.

Preferably, the shell is an aqueous phase.

Advantageously, the shell is transparent. This transparency of the shell is particularly advantageous as it gives a composition according to the invention an impression that the droplets (or core) of fatty phase do not touch and are perfectly suspended in the continuous aqueous phase.

Advantageously, the shell has a homogeneous thickness. By “homogeneous thickness,” in the sense of the present invention, we mean to designate capsules whose shell thickness varies according to a standard deviation less than or equal to 10%, preferably less than or equal to 5%.

In the context of the present description, “gelled envelope” means an external phase surrounding the core of the capsules, and comprising at least one compound in the gelled state or in the form of a gel.

Typically, the shell is a hydrogel of at least one gelled polyelectrolyte and/or at least one thermosensitive hydrophilic gelling agent and/or is a shell deriving from a complex coacervation reaction between two oppositely charged polymers capable of coacervating.

Preferably, the shell comprises at least one gelled polyelectrolyte and/or at least one thermosensitive hydrophilic gelling agent, and/or at least two oppositely charged polymers capable of coacervating, and preferably at least one polyelectrolyte.

The gelled envelope is generally formed by a monolayer of a homogeneous material.

For obvious reasons, the thickness of the shell of the capsules according to the invention is advantageously adjusted to provide satisfaction in terms of mechanical resistance, sprayability, and sensory quality. This thickness of the shell of the capsules is also advantageous as it intervenes in the Ri/Rc ratio, and gives the capacity to a composition according to the invention to easily modulate the rate of fatty phase while maintaining the required volume fraction of capsules.

The invention thus relies on an easy means of decoupling the volume fraction of the capsules from the rate of fatty phase, notably by adjusting the thickness of the shell.

Thus, preferably, the shell of the capsules has a thickness between 62.5 microns and 1,200 microns, preferably between 100 microns and 1,000 microns, better still between 200 microns and 750 microns, especially between 300 microns and 500 microns, or even between 100 microns and 200 microns.

In particular, the shell of the capsules has a thickness greater than 80 microns, preferably greater than 120 microns, and especially greater than 170 microns.

The presence of a shell with a minimum thickness advantageously allows the content of gelled polyelectrolyte(s), thermosensitive hydrophilic gelling agent(s), and/or oppositely charged polymers capable of coacervating to be adjusted, in particular reduced. This allows compositions according to the invention with optimized properties in terms of sprayability and sensory quality to be accessed, while ensuring satisfactory mechanical resistance of the capsules.

When transparent, the presence of a shell with a minimum thickness is also advantageous as it also contributes to the aforementioned visual impression that the droplets (or core) of fatty phase do not touch and are perfectly suspended in the continuous aqueous phase.

Preferably, the shell of the capsules has a thickness different from the values between 80 microns and 200 microns, preferably between 100 microns and 180 microns, better still between 120 microns and 170 microns.

Preferably, the shell of the capsules is a gelled envelope comprising a gel containing water and at least one polyelectrolyte advantageously chosen among proteins, natural polysaccharides, and polyelectrolytes reactive to multivalent ions, and their mixtures.

By “polyelectrolyte reactive to polyvalent ions,” we mean, in the sense of the present invention, a polyelectrolyte capable of transitioning from a liquid state in an aqueous solution to a gelled state under the effect of contact with a gelling solution containing multivalent ions, such as ions of an alkaline earth metal chosen for example among calcium ions, barium ions, magnesium ions.

In the liquid state, the individual chains of polyelectrolyte are substantially free to flow relative to each other. An aqueous solution of 2% by mass of polyelectrolyte then exhibits a purely viscous behavior at the shear gradients characteristic of the shaping process. The viscosity of this zero-shear solution is between 50 mPa·s and 10,000 mPa·s, advantageously between 3,000 mPa·s and 7,000 mPa·s. This viscosity at the shear gradients characteristic of the flows involved in the manufacture of the capsules is for example measured using a stress or strain-imposed rheometer at the manufacturing temperature, 25° C. for example. For the measurements, a cone-plate geometry with a diameter between 10 and 50 mm, and a cone angle of 1° maximum will be used.

The individual chains of polyelectrolyte in the liquid state advantageously have a molar mass greater than 65,000 g/moles.

In the gelled state, the individual chains of polyelectrolyte form, with the multivalent ions, a coherent three-dimensional network that retains the liquid core and prevents its flow. The individual chains are retained relative to each other and cannot flow freely relative to each other. In this state, the viscosity of the formed gel is infinite.

The three-dimensional polyelectrolyte gel contained in the envelope traps water and the surfactant agent when present.

Advantageously, the polyelectrolyte is chosen among polysaccharides, synthetic polyelectrolytes based on acrylates (sodium, lithium, potassium, or ammonium polyacrylate, or polyacrylamide), synthetic polyelectrolytes based on sulfonates (sodium poly(styrene sulfonate), for example). More particularly, the polyelectrolyte is chosen among alkaline earth alginates, such as sodium alginate or potassium alginate, gellan, or pectin.

Preferably, the polyelectrolyte is chosen among a protein such as collagen; a natural polysaccharide, such as heparan sulfate; a polyelectrolyte reactive to multivalent ions, notably a polysaccharide reactive to multivalent ions, such as alginate, pectin, particularly Low Methoxyl pectin, carrageenan, particularly kappa and iota-carrageenan, gellan gum, diutan gum, furcellaran, or one of their derivatives, and their mixtures; and preferably a polyelectrolyte reactive to multivalent ions.

According to one embodiment of the invention, the polyelectrolyte is sodium alginate.

Alginates are produced from brown algae called “laminaria,” referred to by the term “seaweed.”

By “thermosensitive gelling agent,” we mean to designate a gelling agent that reacts to heat, and notably is a solid gelling agent at room temperature and liquid at a temperature above 40° C., preferably above 50° C.

Preferably, the thermosensitive hydrophilic gelling agent is chosen among agar, kappa carrageenan, konjac, gelatin, and their mixtures.

Preferably, the thermosensitive hydrophilic gelling agent is not a hydrophilic gelling agent capable of gelling in the presence of at least one salt.

The oppositely charged polymers capable of coacervating comprise at least one cationic polymer and at least one anionic polymer.

In view of the above, these oppositely charged polymers capable of coacervating are advantageously hydrophilic.

According to a particular embodiment, the anionic polymer is hydrophilic and the cationic polymer is lipophilic, and thus present in the fatty phase, or vice versa. This embodiment is notably illustrated in WO2012120043.

The oppositely charged polymers capable of coacervating can be chosen among the couples “gum arabic (or acacia gum)/gelatin,” “albumin/alginate,” or “gelatin/alginate.”

Of course, the person skilled in the art will ensure to choose the polyelectrolyte(s) and/or thermosensitive hydrophilic gelling agent(s) and/or oppositely charged polymers capable of coacervating and/or their quantity in such a way that the advantageous properties of a composition according to the invention are not or substantially not altered by the envisaged addition. These adjustments fall within the general knowledge of the person skilled in the art.

A composition according to the invention can advantageously comprise between 0.25% and 5%, preferably between 0.5% to 3%, and better still between 0.75% and 1%, by weight of polyelectrolyte(s) and/or thermosensitive hydrophilic gelling agent(s) and/or oppositely charged polymers capable of coacervating, relative to the total weight of the shell.

Advantageously, the shell, notably when represented by a gelled envelope as described above, can further comprise at least one surfactant agent.

The surfactant agent is advantageously an anionic surfactant, a nonionic surfactant, a cationic surfactant, or a mixture thereof. The molecular weight of the surfactant agent is advantageously between 150 g/mol and 10,000 g/mol, preferably between 250 g/mol and 1,500 g/mol.

In the case where the surfactant is an anionic surfactant, it is for example chosen among alkyl sulfates, alkyl sulfonates, alkylaryl sulfonates, alkaline alkyl phosphates, dialkyl sulfosuccinates, alkaline earth salts of saturated or unsaturated fatty acids, or a mixture thereof. These surfactants advantageously have at least one hydrophobic hydrocarbon chain with a number of carbons greater than 5, or even 10, and at least one hydrophilic anionic group, such as a sulfate, sulfonate, or carboxylate linked to one end of the hydrophobic chain.

In the case where the surfactant is a cationic surfactant, it is for example chosen among alkylpyridinium or alkylammonium halide salts, such as n-ethyldodecylammonium chloride or bromide, cetylammonium chloride or bromide (CTAB), or a mixture thereof. These surfactants advantageously have at least one hydrophobic hydrocarbon chain with a number of carbon atoms greater than 5, or even 10, and at least one hydrophilic cationic group, such as a quaternary ammonium cation.

In the case where the surfactant is a nonionic surfactant, it is for example chosen among polyoxyethylene and/or polyoxypropylene derivatives of fatty alcohols, fatty acids, or alkylphenols, arylphenols, or among alkylglucosides, polysorbates, cocamides, or a mixture thereof.

According to one embodiment of the invention, the surfactant agent is sodium lauryl sulfate (SLS or SDS) or sodium lauryl glutamate.

The mass content of surfactant agent(s) in the shell is advantageously greater than 0.001%, preferably greater than 0.1%, particularly between 0.001% and 1%, and better still between 0.01% and 0.5%, relative to the total weight of the shell.

### Core

The capsules comprise a liquid core or at least partially gelled or at least partially thixotropic, and preferably liquid.

According to a first particular embodiment, the fatty phase of the core of the capsules according to the invention is monophasic and thus comprises only one fatty phase.

According to a second particular embodiment, the fatty phase of the core of the capsules according to the invention is at least biphasic; such a fatty phase is thus in the form of an inverse emulsion (or “water-in-oil emulsion”) wherein the dispersed phase is in the form of microscopic droplets.

According to a first embodiment, the capsule is a so-called “simple” capsule, meaning that the core is monophasic, i.e., consists of a single fatty phase, the fatty phase being placed in whole or in part in contact with the shell. A simple capsule is for example a capsule as described in WO2010/063937.

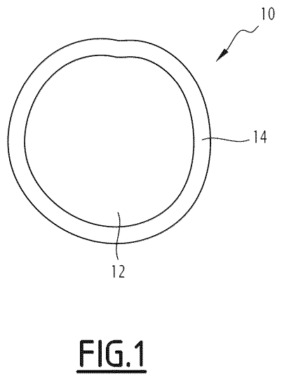

According to a first variant, a simple capsule comprises two distinct phases, namely a fatty phase in the form of a single droplet and an external gelled phase surrounding the fatty phase. This first variant of a simple capsule is illustrated in FIG. 1 (10: capsule; 14: shell; 12: fatty phase).

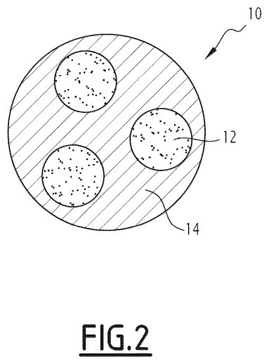

According to a second variant, a simple capsule comprises two distinct phases, namely a fatty phase in the form of several droplets and an external gelled phase surrounding all the droplets of fatty phase. This second variant of a simple capsule is illustrated in FIG. 2 (10: capsule; 14: shell; 12: fatty phase).

Alternatively, in the simple capsules described above, the fatty phase can itself be in the form of an inverse emulsion.

According to another embodiment, the capsule is a so-called “complex” capsule, meaning that the core comprises an intermediate fatty phase, the intermediate fatty phase being placed in whole or in part in contact with the shell, and at least one internal droplet of an internal phase disposed in the intermediate fatty phase.

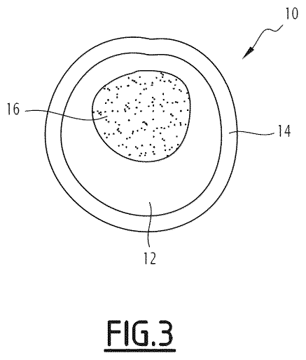

According to a first variant, a complex capsule is such that the core comprises a single intermediate droplet of an intermediate fatty phase, the intermediate fatty phase being placed in whole or in part in contact with the shell, and at least one internal droplet of an internal phase disposed in the intermediate droplet and immiscible with the intermediate droplet. This second variant of a simple capsule is illustrated in FIG. 3 (10: capsule; 14: shell; 12: fatty phase; 16: internal phase).

According to a second variant (not illustrated), a complex capsule is such that the core comprises several intermediate droplets of intermediate fatty phase, the intermediate fatty phase being placed in whole or in part in contact with the shell, and each intermediate droplet comprises at least one internal droplet of an internal phase disposed in each intermediate droplet and immiscible with the intermediate droplet.

The core of such complex capsules can thus comprise an intermediate fatty phase wherein a single droplet of internal phase or a plurality of internal phase droplets is found.

Alternatively, in the complex capsules described above, the fatty phase can itself be in the form of an inverse emulsion.

For obvious reasons, the continuous aqueous phase and the fatty phase are immiscible. By “immiscible” or “non-miscible” in the sense of the present invention, we mean to designate that the solubility of a first phase in a second phase is advantageously less than 5% by mass, and vice versa.

For obvious reasons, in complex capsules, the intermediate fatty phase and the internal phase are immiscible.

Thus, according to a first variant of complex capsules, the intermediate phase is a fatty phase and the internal phase is an aqueous phase. According to a second variant, the intermediate phase is a first fatty phase and the internal phase is a second fatty phase immiscible with the first fatty phase. Non-miscible oil couples are notably described in FR3063893.

Each internal droplet of a complex capsule advantageously has a substantially spherical shape. Alternatively, the shape of the internal droplet is different from a spherical shape, for example elliptical or lenticular.

The internal droplets of complex capsules are advantageously macroscopic. The internal droplets of complex capsules advantageously have a size (or diameter) greater than 150 μm, and notably greater than or equal to 100 μm, preferably greater than or equal to 200 μm, but in all cases necessarily less than the diameter of the core of the capsules. The determination of the size of the capsules or droplets falls within the general knowledge of the person skilled in the art and can notably be measured by a method using the image processing software “Image J.”

The sum of the volumes of the or each internal droplet is advantageously from 5% to 65% of the total volume of the core, and preferably from 10% to 55% of the volume of the core.

In view of the above, in a composition according to the invention, the capsules can comprise a liquid core or at least partially gelled or at least partially thixotropic, the core being monophasic or comprising an intermediate droplet of fatty phase, and at least one, preferably a single, internal droplet of an internal phase disposed in the intermediate droplet.

The minimum volume of the core is advantageously between 5% and 80%, preferably between 10% and 70%, better still between 20% and 60%, and especially between 30% and 50%, relative to the total volume of the capsule.

Preferably, the core of the capsules has a diameter between 125 μm and 1,500 μm, better still between 250 μm and 1,200 μm, and especially between 500 μm and 1,000 μm.

In the case of complex capsules, the core is understood as the intermediate phase and the internal phase.

The capsules of a composition according to the invention represent a volume fraction between 50% and 70% relative to the total volume of the composition.

As previously indicated, it is this high volume fraction in capsules that ensures their suspension in the continuous aqueous phase.

Preferably, the volume fraction of the capsules is between 55% and 65%, and preferably between 57% and 63% relative to the total volume of the composition.

In particular, the inventors observed that the nature and/or content of gelled polyelectrolyte(s) and/or thermosensitive hydrophilic gelling agent(s) forming the shell of the capsules allows the mechanical resistance (or compression resistance), and thus the deformability, of the capsules to be modulated, thus allowing the volume fraction of the capsules to be further increased relative to the total volume of the composition.

Thus, and as previously indicated, the capsules of a composition according to the invention capsules comprise a ratio Ri/Rc between 0.5 and 0.8, preferably between 0.6 and 0.8, and better still between 0.6 and 0.7, wherein:
Ri corresponds to the radius of the core of the capsules; and
Rc corresponds to the radius of the capsule, i.e., resulting from the sum of the radius of the core of the capsules and the thickness of the shell.

In cases where the capsules are complex capsules, Rc also comprises the radius of the internal droplets of the internal phase.

Preferably, the capsules according to the invention comprise a ratio (e)/Ri between 0.2 and 1, preferably between 0.3 and 0.8, and especially between 0.4 and 0.7, wherein:
(e) corresponds to the thickness of the shell; and
Ri corresponds to the radius of the core of the capsules.

As it appears from example 4 below, these values of the Ri/Rc ratio or the (e)/Ri ratio correspond to a satisfactory and non-obvious compromise between mechanical resistance, sprayability, and sensory quality.

According to a particular embodiment, a composition according to the invention can comprise between 45% and 85%, preferably between 50% and 75%, and better still between 55% and 70%, by weight of capsules relative to the total weight of the composition.

Advantageously, a composition according to the invention can comprise between 5% and 40%, preferably between 10% and 30%, and better still between 15% and 20%, by weight of fatty phase relative to the total weight of the composition.

### Active Agents

The core of the capsules comprises at least one active agent.

Preferably, an active agent is different from a fragrance agent. In other words, an active agent is not a fragrance agent.

For obvious reasons, an active agent according to the invention is a lipophilic agent, i.e., soluble or dispersible in an organic solvent, particularly an oil.

In the context of the present description, “active agent” means a compound having a beneficial physiological effect on the element on which it acts. It aims, for example, to protect, maintain in good condition, care for, heal, flavor, or color.

The active agent is advantageously a cosmetic, dermo-pharmaceutical, pharmaceutical, or food agent. The core of the capsules preferably comprises at least one active agent chosen among at least one cosmetic active agent, at least one biologically active product, and their mixtures, and preferably at least one cosmetic active agent.

The core of the capsules can contain the active agent in the form of pure liquid, or a solution of the active agent in an organic solvent, or a suspension of the active agent in an organic solvent. Preferably, the organic solvent is at least one oil.

When the active agent is a cosmetic agent, it can be chosen among moisturizing agents, healing agents, vitamins (e.g., Bakuchiol), depigmenting agents, pigments, nacres, hair actives, UV filters, desquamating agents, antioxidants, actives stimulating the synthesis of dermal and/or epidermal macromolecules, dermo-relaxing agents, anti-perspirant agents, soothing agents, emollients, softening agents, anti-wrinkle agents, protective/anti-radical agents, anti-cellulite, firming, shaping, draining agents, anti-aging agents, anti-irritant agents, tightening/smoothing agents, whitening agents, self-tanning agents, exfoliating agents, agents stimulating cell renewal or stimulating cutaneous microcirculation, anti-dandruff agents, and their mixtures.

Dermo-pharmaceutical agents more particularly designate agents acting at the skin level.

When the active agent is a pharmaceutical agent, it is advantageously chosen among anticoagulants, anti-thrombotic agents, anti-mitotic agents, anti-proliferation, anti-adhesion, anti-migration agents, cell adhesion promoters, growth factors, antiparasitic molecules, anti-inflammatory agents, angiogenic agents, angiogenesis inhibitors, vitamins, hormones, proteins, antifungals, antimicrobial molecules, antiseptics, or antibiotics, and their mixtures.

Food agents are advantageously flavors, food oils, such as olive oil, soybean oil, grape seed oil, sunflower oil, or any other oil extracted from plants, and their mixtures. A flavor can be chosen among natural flavors; these are notably extracts from natural sources, such as fruits, vegetables, herbs, and spices, obtained by different methods, such as, for example, distillation, extraction, or fermentation. A flavor can be chosen among artificial flavors. In particular, a flavor or a food oil can be represented by an essential oil.

In particular, a composition according to the invention is dedicated to the care of keratinous materials, particularly the skin, wherein case the composition comprises at least one cosmetic active agent chosen among moisturizing agents, vitamins, healing agents, anti-wrinkle agents, depigmenting agents, desquamating agents, antioxidants, active agents stimulating the synthesis of dermal and/or epidermal macromolecules, dermo-relaxing agents, anti-perspirant agents, soothing agents, emollients, softening agents, protective/anti-radical agents, anti-cellulite, firming, shaping, draining agents, anti-aging agents, anti-irritant agents, tightening/smoothing agents, whitening agents, self-tanning agents, exfoliating agents, agents stimulating cell renewal or stimulating cutaneous microcirculation, anti-dandruff agents, and their mixtures.

As such cosmetic active agents usable in the present invention, one can cite those described in the patent application filed under no. FR2203882.

In particular, a composition according to the invention is dedicated to the makeup of keratinous materials, particularly the skin, in which case the composition comprises at least pigments and/or nacres as an active agent.

By “pigment,” we mean a coloring chemical substance insoluble in the phase wherein the pigment is present. By “insoluble,” we mean that the solubility at 20° C. of the pigment in the phase wherein the pigment is present is less than 1 g/L, notably less than 0.1 g/L, preferably less than 0.001 g/L.

Each pigment can independently be an organic, inorganic, or hybrid organic-inorganic pigment. These are typically inorganic pigments.

The coloration conferred by a composition according to the invention can, for example, be measured by spectrocolorimetry and/or spectrophotocolorimetry.

Coverage corresponds to the ability of a composition to “mask the skin”/to “hide imperfections.” The coverage of a composition according to the invention can be measured according to the method described in the patent application filed under no. FR2203883.

As pigments, one can notably cite titanium dioxide, zinc dioxide, zirconium or cerium oxides, as well as iron or chromium oxides, manganese violet, ultramarine blue, chromium hydrate, and ferric blue, and their mixtures. Preferred mineral pigments are iron oxides, notably red iron oxide, yellow iron oxide, brown iron oxide, black iron oxide, titanium dioxide, and their mixtures.

The pigment is preferably an iron oxide, notably red iron oxide, yellow iron oxide, brown iron oxide, black iron oxide, and their mixtures.

Each pigment can be an untreated pigment or a treated pigment. In the sense of the application, by “treated pigment,” we mean a pigment that has been treated with an additive improving its dispersibility within the phase comprising it. By “untreated pigment” or “not treated pigment,” we mean a pigment that has not been treated with such an additive.

According to a first alternative, the pigment used is an untreated and unground pigment (pigment used “as is”).

According to a second alternative, the pigment used has undergone a preliminary treatment to make it more easily dispersible during the formulation of the pigment, i.e., notably more easily dispersible in the considered phase. This preliminary treatment consists of grinding the pigment and/or pre-treating it with an additive improving its dispersibility before formulating it in the form of a series of colored particles.

As pigments, and additive(s) usable in the present invention, one can cite those described in the patent application filed under no. FR2203883.

By “nacres,” we mean colored particles of any shape, iridescent or not, notably produced by certain mollusks in their shell or synthesized and presenting a color effect by optical interference.

As nacres usable in the present invention, one can cite those described in the patent application filed under no. FR2112591.

In particular, a composition according to the invention is dedicated to the care of keratinous fibers, particularly hair and/or scalp (or hair care composition), in which case the composition comprises at least one hair active agent as an active agent.

As hair active agents according to the invention, one can cite those commonly used for hair and/or scalp care compositions. In particular, as a hair active agent according to the invention, one can cite surfactants; fixing polymers, notably those listed in applications FR2964565 or US2013289080; conditioning agents, notably chosen among cationic conditioning agents; fatty substances different from the oils and lipophilic gelling agents mentioned above; polysaccharides, notably chitosans or their derivatives; UV agents (or UV filters), notably those described in FR3041251; fillers such as resins or clays; peptizing agents; acid agents; alkaline agents; reducing agents; oxidizing agents; direct dyes or oxidation dyes; and their mixtures.

As hair active agents usable in the present invention, one can cite those described in the patent application filed under no. FR2206462.

In particular, a composition according to the invention is dedicated to sun protection (or sun composition), in which case the composition comprises at least UV filters as an active agent. Such a sun composition allows a keratinous material, particularly the skin, to be protected against the rays of the sun, particularly at least against UVA and/or UVB rays.

The Sun Protection Factor (SPF) of a sun composition is a measure of its effectiveness. It indicates the protective power of a product against sunburn. A sun composition according to the invention thus advantageously has an SPF greater than or equal to 15, preferably greater than or equal to 30, and better still greater than or equal to 50. The measurement of the SPF falls within the general knowledge of the person skilled in the art.

As UV filters, one can notably cite organic UV filters, inorganic UV filters, and their mixtures.

As an illustration of organic UV filters and in a non-limiting way, one can cite:
anthranilates, particularly menthyl anthranilate;
benzophenones, particularly benzophenone-1, benzophenone-3 or oxybenzone, benzophenone-5, benzophenone-6, benzophenone-8, benzophenone-9, benzophenone-12, and preferably Benzophenone-2 (Oxybenzone), or Benzophenone-4 (Uvinul MS40® available from B.A.S.F.);
benzylidene camphors, particularly 3-benzylidene camphor, benzylidene camphor sulfonic acid, camphor benzalkonium methosulfate, polyacrylamidomethylbenzylidene camphor, terephthalylidene di-camphor sulfonic acid, and preferably 4-methylbenzylidene camphor (Eusolex 6300® available from Merck);
benzimidazoles, particularly benzimidazilate (Neo Heliopan AP® available from Haarmann and Reimer), or phenylbenzimidazole sulfonic acid (PARSOL HS® available from DSM);
benzotriazoles, particularly drometrizole trisiloxane, or methylene bis-benzotriazolyltetramethylbutylphenol (Tinosorb M® available from Ciba);
cinnamates, particularly cinoxate, DEA methoxycinnamate, diisopropyl methylcinnamate, dimethoxycinnamate ethylhexanoate glycerol, isopropyl methoxycinnamate, isoamyl cinnamate, Kaempferia galanga root extract (TEGO GALANGA from EVONIK containing 98% ethyl-p-methoxycinnamate) and preferably octylmethoxycinnamate (Parsol MCX® available from Hoffmann La Roche),
diphenyl acrylates, particularly ethocrylene (Uvinul N35® available from B.A.S.F.), or octocrylene (Uvinul 539® available from B.A.S.F.) or Ethylhexyl methoxycrylene (SOLASTAY available from HALLSTAR)
dibenzoylmethanes, particularly butyl methoxydibenzoylmethane (Parsol 1789®); imidazolines, particularly ethylhexyl dimethoxybenzylidene dioxoimidazoline;
PABAs, particularly ethyl Dihydroxypropyl PABA, ethylhexyldimethyl PABA, glyceryl PABA, PABA, PEG-25 PABA, or ethyl PABA (benzocaine),
triazines, particularly anisotriazine (Tinosorb S® available from Ciba) or diethylhexylbutamido-triazone (Uvasorb HEB® available from 3V Sigma), ethylhexyltriazone (Uvinul T150® available from B.A.S.F.), Tris-Biphenyl Triazine (Tinosorb 2AB available from BASF),
benzoates, particularly N-hexyl 2-(4-diethylamino-2-hydroxybenzoyl)benzoate (Uvinul A+ available from BASF) or in mixture with octyl methoxycinnamate (Uvinul A+B available from BASF),
benzalmalonates, particularly Polysilicone-15 (Parsol SLX available from DSM),
   benzoxazoles, particularly 2,4-Bis[4-[5-(1,1-dimethylpropyl)benzoxazol-2-yl]phenylimino]-6-[(2-ethylhexyl)imino]-1,3,5-triazine, (Uvasorb K2A available from Sigma 3V),
salicylates, particularly dipropylene glycol salicylate, ethylhexyl salicylate, homosalate, butyloctyl Salicylate (HALLBRITE BHB available from HALLSTAR or TEA salicylate; and
their mixtures.

As UV filters usable in the present invention, one can cite those described in the patent application filed under no. FR2203887.

The characteristics of a composition according to the invention, particularly those of the capsules, are such that they allow high contents of active agent(s), without prejudice to the kinetic stability of the composition and in particular without prejudice to the suspension of the capsules in the continuous aqueous phase.

Thus, the fatty phase of a composition according to the invention can advantageously comprise from 0.01% to 30%, preferably from 0.1% to 20%, and better still from 1% to 10%, by weight of active agent(s) relative to the total weight of the composition.

Also, the composition can advantageously comprise from 0.1% to 100%, preferably from 1% to 90%, particularly from 2.5% to 80%, or even from 5% to 70%, especially from 0.1% to 30%, and better still from 1% to 10%, by weight of active agent(s) relative to the total weight of the fatty phase of the capsules.

### Oils

According to a particular embodiment, the core of the capsules can further comprise at least one oil, different from the food oils described previously.

By “oil,” we mean a fatty substance liquid at room temperature.

As oils usable in a dispersion of the invention, one can cite for example:
hydrocarbon oils of vegetable origin, such as hydrogenated jojoba oil, hydrogenated sunflower oil, hydrogenated castor oil, hydrogenated copra oil;
hydrocarbon oils of animal origin, such as perhydrosqualene and squalane;
synthetic esters and ethers, notably of fatty acids, such as oils of formulas R1COOR2 and R1OR2 wherein R1 represents the residue of a fatty acid in C8 to C29, and R2 represents a hydrocarbon chain, branched or not, in C3 to C30, such as Purcellin oil, isononyl isononanoate, isodecyl neopentanoate, isopropyl myristate, ethyl-2-hexyl palmitate, octyl-2-dodecyl stearate, octyl-2-dodecyl erucate, isostearyl isostearate; hydroxylated esters, such as isostearyl lactate, octylhydroxystearate, octyldodecyl hydroxystearate, diisostearyl malate, triisocetyl citrate, heptanoates, octanoates, decanoates of fatty alcohols; polyol esters, such as propylene glycol dioctanoate, neopentyl glycol diheptanoate, and diethylene glycol diisononanoate; and pentaerythritol esters, such as pentaerythrityl tetrabehenate (DUB PTB) or pentaerythrityl tetraisostearate (Prisorine 3631);
linear or branched hydrocarbons, of mineral or synthetic origin, such as paraffin oils, volatile or not, and their derivatives, Vaseline, polydecenes, hydrogenated polyisobutene, such as Parleam oil;
silicone oils, such as polymethylsiloxanes (PDMS) volatile or not with linear or cyclic silicone chain, liquid or pasty at room temperature, notably cyclopolydimethylsiloxanes (cyclomethicones), such as cyclohexasiloxane and cyclopentasiloxane; polydimethylsiloxanes (or dimethicones) including alkyl, alkoxy, or phenyl groups, during or at the end of the silicone chain, groups having from 2 to 24 carbon atoms; phenylated silicones, such as phenyltrimethicones, phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, and polymethylphenylsiloxanes;
fatty alcohols having from 8 to 26 carbon atoms, such as cetyl alcohol, stearyl alcohol, and their mixture (cetylstearyl alcohol), or even octyldodecanol;
partially hydrocarbonated and/or siliconized fluorinated oils such as those described in document JP-A-2-295912;
and their mixtures.

According to another preferred embodiment, the fatty phase does not comprise silicone oil, and preferably does not comprise polydimethylsiloxane (PDMS).

Of course, the person skilled in the art will ensure to choose the possible oils and/or their quantity in such a way that the advantageous properties of a composition according to the invention are not or substantially not altered by the envisaged addition. These adjustments fall within the general knowledge of the person skilled in the art.

According to one embodiment, a dispersion according to the invention comprises between 0% and 99.90%, particularly between 10% and 99%, preferably between 20% and 97.5%, and especially between 30% and 95%, by weight of oil(s) relative to the total weight of the fatty phase.

### Lipophilic Gelling Agents

According to a particular embodiment, the core of the capsules can further comprise at least one lipophilic gelling agent, notably when the core is at least partially gelled or at least partially thixotropic.

A lipophilic gelling agent, i.e., soluble or dispersible in the fatty phase, can be chosen among organic or mineral, polymeric or molecular gelling agents; solid fatty substances at ambient temperature and pressure, notably chosen among waxes, pasty fatty substances, butters; and their mixtures, and preferably among polymeric gelling agents. Such lipophilic gelling agents are notably described in WO2019002308.

Among the lipophilic gelling agents that can be used in the present invention, one can cite dextrin esters and fatty acids, such as dextrin palmitates. Among dextrin esters and fatty acid(s), one can, for example, cite dextrin palmitates, dextrin myristates, dextrin palmitates/ethylhexanoates, and their mixtures. One can notably cite dextrin esters and fatty acid(s) marketed under the names Rheopearl® KL2 (INCI: dextrin palmitate), Rheopearl® TT2 (INCI: dextrin palmitate ethylhexanoate), and Rheopearl® MKL2 (INCI: dextrin myristate) by Miyoshi Europe, also dextrin palmitate marketed by The Innovation Company.

One can also cite THIXCIN® R from Elementis Specialties (INCI: Trihydroxystearin), OILKEMIA™ 5S polymer from Lubrizol (INCI: Caprylic/Capric Triglyceride (and) Polyurethane-79), Oilkemia™ 5S CC polymer, (INCI: INCI: Caprylic/Capric Triglyceride (and) Hydrogenated Poly(C6-20 Olefin) (and) HDI/Trimethylol Hexyllactone Crosspolymer), Estogel M from PolymerExpert (INCI: CASTOR OIL/IPDI COPOLYMER & CAPRYLIC/CAPRIC TRIGLYCERIDE), EMC30 (INCI: Caprylic/Capric Triglyceride (and) Castor Oil/IPDI Copolymer), Hydrogenated Castor Oil/Sebacic Acid Copolymer as well as its derivatives, notably marketed respectively under the names Estogel Green (or Estogel G) and Estogel Green 40 by PolymerExpert, and their mixtures.

Advantageously, a lipophilic gelling agent is a thermosensitive gelling agent.

According to the invention, a composition according to the invention can comprise from 0.5% to 30%, preferably from 1% to 25%, particularly from 1.5% to 20%, better still from 2% to 15%, and especially from 5% to 12%, by weight of lipophilic gelling agent(s) relative to the total weight of the fatty phase.

Of course, the person skilled in the art will ensure to choose the possible lipophilic gelling agent(s) and/or their quantity in such a way that the advantageous properties of a composition according to the invention are not or substantially not altered by the envisaged addition. These adjustments fall within the general knowledge of the person skilled in the art.

### Monodispersity

Preferably, the capsules present a monodisperse distribution.

This monodispersity advantageously contributes to the aforementioned steric suspension and sprayability. Indeed, the inventors observed that with a composition comprising polydisperse capsules, the smaller capsules tend to occupy the spaces between the larger capsules. This phenomenon leads to the density in capsules being locally increased and thus mechanically to a creaming of the capsules. These disadvantages impact the visual of the composition and the homogeneity during application, or even the sprayability of the composition. A solution to counter these disadvantages would be to add in the continuous aqueous phase at least one hydrophilic gelling agent, such as those described previously for the purpose of increasing viscosity. This solution is, however, not optimal given the aforementioned disadvantages with prior art dispersions.

By “monodisperse,” we mean to designate, in the sense of the present invention, a composition whose polydispersity in capsule size, measured by the coefficient of variation Cy consisting of the ratio of the standard deviation to the mean, is less than 10%, and notably from 1% to 10%.

This ratio can be measured, for example, based on the diameters measured on at least seven capsules using the image processing software “Image J,” based on a snapshot of the capsules taken in top view with a digital camera.

Alternatively, the polydispersity in capsule mass can be calculated, based on at least 50 measurements of the mass of 50 complex capsules carried out using a Mettler-Toledo balance with a precision of 0.1 mg.

### Additional Compound(s)

According to the invention, a dispersion according to the invention, in particular the continuous phase and/or the phase constituting the shell and/or the fatty phase, may comprise at least one additional compound different from the oils, hydrophilic and lipophilic gelling agents, and active agents mentioned above.

The dispersions according to the invention can thus further comprise powders; reflective particles, particularly glitter, notably as defined in FR3082731; coloring agents, notably chosen among water-soluble or not, lipid-soluble or not, organic or inorganic coloring agents, optical effect materials, liquid crystals, and their mixtures; fillers, notably as described in FR1755907; emulsifying and/or non-emulsifying silicone elastomers, notably as described in EP2353577; “soft focus” fillers; texture agents, notably polyethylene glycols (e.g., marketed under the name Carbowax) or glycerin; fragrance agents; preservatives; humectants; stabilizers; chelating agents; pH modifiers, osmotic strength modifiers, and/or refractive index modifiers, etc.; or any usual cosmetic additive; and their mixtures.

### Additional Dispersed Phase

A composition according to the invention can further comprise:
capsules comprising a shell and a core comprising at least one aqueous phase, optionally the aqueous core further comprises at least one hydrophilic active agent; and/or
full (or matrix) macroscopic beads.

By beads, we mean to designate, in the sense of the present invention, a macroscopic spherical element devoid of a shell; the constitutive phase can also comprise at least one active agent.

In the context of the present invention, full beads can indifferently be designated by the expression “beads,” “balls,” “spheres,” or “additional dispersed phase.”

A composition according to the invention can further comprise full macroscopic hydrophilic or lipophilic beads.

A composition according to the invention can further comprise full macroscopic beads whose constitutive phase is a direct or inverse emulsion.

A full macroscopic hydrophilic bead comprises water and at least one gelled polyelectrolyte and/or at least one thermosensitive hydrophilic gelling agent, notably as described previously.

A full macroscopic lipophilic bead comprises at least one lipophilic gelling agent and optionally at least one oil, notably as described previously.

Such beads allow the presence in a composition according to the invention of a hydrophilic or lipophilic active agent incompatible with other ingredients present in the composition.

As a lipophilic active agent, one can cite those described previously.

As a hydrophilic active agent, one can cite proteins or protein hydrolysates, amino acids, polyols notably in C2 to C10, urea, allantoin, sugars and sugar derivatives, water-soluble vitamins, starch, bacterial or plant extracts, such as Aloe Vera, and their mixtures.

### PARTICULAR EMBODIMENTS

A composition according to the invention can comprise a single population of capsules among the different variants of simple and complex capsules described previously.

Alternatively, a composition according to the invention can comprise at least two different populations of capsules chosen among the different variants of simple and complex capsules described previously.

Thus, according to a first variant of embodiment, a composition according to the invention can comprise at least two populations of capsules with different Ri/Rc ratios, of identical or different sizes, as illustrated in example 5 below.

According to a second variant of embodiment, a composition according to the invention can comprise a first population of simple capsules and a second population of complex capsules, of identical or different sizes.

Such variants of embodiment can further comprise full beads as described previously.

All these variants are advantageous as they notably allow compositions comprising macroscopic elements of different densities and/or sizes and/or mechanical resistance to be accessed, thus potentially leading to different visual/aesthetic effects and/or cosmetic effects and/or sensory effects.

### Preparation Process

The preparation of a continuous aqueous phase of a composition according to the invention is part of the general knowledge of a person skilled in the art.

The preparation of capsules of a composition according to the invention is also part of the general knowledge of a person skilled in the art. As an illustrative example, by means of the invention, the capsules of a composition can be obtained using non-micro/milli-fluidic manufacturing processes, such as agitation with Rayneri, or using micro/milli-fluidic manufacturing processes, for example those described in WO2010063937, FR2964017, WO2012089820, WO2021037999, WO2022106361, EP2292752, EP0525731, EP1020177, EP1025842, or in patent applications filed under numbers FR2314864 and FR2314873.

The preparation of solid beads is also part of the general knowledge of a person skilled in the art. As an illustrative example, by means of the invention, the beads can be obtained using any process known to a person skilled in the art, for example those described in BRPI1001438 or FR2645439.

### Uses

The compositions according to the invention can notably be used in the cosmetic, dermo-pharmaceutical, pharmaceutical, or food fields.

A person skilled in the art will know how to adjust the grade of raw materials with regard to the field of use considered.

According to a preferred embodiment, a composition according to the invention can be used in the cosmetic field and is therefore a cosmetic composition.

According to another preferred embodiment, a composition according to the invention can be used in the food field and is therefore a food or nutritional composition.

As an illustrative example, by means of the invention, such a composition can be a dietary supplement.

Preferably, a composition according to the invention is directly usable, at the end of the aforementioned preparation processes, as a composition, notably cosmetic.

They can comprise, in addition to the aforementioned ingredients or compounds, at least one physiologically acceptable medium.

The physiologically acceptable medium is generally adapted to the nature of the support on which the composition is to be applied, as well as to the form wherein the composition is to be conditioned.

According to one embodiment, the physiologically acceptable medium is directly represented by the continuous aqueous phase as described above.

In the context of the invention, and unless otherwise mentioned, “physiologically acceptable medium” means a medium appropriate for cosmetic applications, and particularly suitable for applying a composition of the invention to keratinous material, notably the skin and/or hair, and more particularly the skin.

The cosmetic compositions of the invention can be, for example, a cream, a lotion, a serum, and a gel for the skin (hands, face, feet, etc.), a foundation (liquid, paste), a preparation for baths and showers (salts, foams, oils, gels, etc.), a hair care product (e.g., hair dyes and bleaches), a cleaning product (lotions, powders, shampoos), a hair care product (lotions, creams, oils), a styling product (lotions, sprays, brilliantines), a shaving product (soaps, foams, lotions, etc.), a product intended to be applied to the lips, a sun product, a self-tanning product, a product for whitening the skin, an anti-aging product. In particular, the cosmetic compositions of the invention can be an anti-aging serum, a youth serum, or a moisturizing serum.

Thus, in view of the above, a dispersion or composition according to the invention is oral or topical, preferably topical, and better still topical on keratinous material, particularly the skin, and better still the facial skin.

In particular, a cosmetic composition according to the invention is dedicated to the care and/or makeup of keratinous material, particularly the skin and/or hair. In particular, a composition according to the invention can be a BB Cream or a CC cream.

In particular, a composition according to the invention is dedicated to the care of keratin fibers, particularly hair and/or scalp (or hair care composition).

In particular, a cosmetic composition according to the invention is dedicated to the sun protection (or sun composition) of keratinous material, particularly the skin.

Advantageously, a composition according to the invention is not a perfuming composition. A composition according to the invention can, however, be perfumed without being perfuming.

The invention also relates to a bottle provided with an application means and a conditioning means, said conditioning means containing a composition as described above.

The present invention also relates to a non-therapeutic cosmetic treatment process of keratinous material, particularly for the care and/or makeup of keratinous material, and notably the skin and/or hair, and more particularly the skin, comprising a step of applying to said keratinous material at least one aforementioned cosmetic composition.

The present invention also relates to the use of a composition according to the invention, for the care and/or makeup of keratinous material, and notably the skin and/or hair, and more particularly the skin.

The present invention also relates to the use of a composition according to the invention, for the sun protection of keratinous material, particularly the skin.

In particular, the present invention relates to the use of a composition according to the invention, to improve the surface appearance of the skin, particularly to hydrate the skin and/or reduce wrinkles and fine lines.

The composition will preferably be applied/used by means of a spraying device (or means).

Throughout the description, the expression “comprising a” must be understood as synonymous with “comprising at least one,” unless specified otherwise. The expressions “between . . . and . . . ”, “from . . . to . . . ”, and “ranging from . . . to . . . ” must be understood as inclusive of the limits, unless specified otherwise.

### EXAMPLES

### Example 1: Preparation of a Continuous Aqueous Phase

The table below indicates the composition of an aqueous phase, dedicated to representing the continuous aqueous phase of the compositions described in examples 6 to 8 below.

| Compound | Mass % |
|---|---|
| Water | SQF* |
| Glycerol | 19.0 |
| Propanediol (Zemea, Dupont | 7.0 |
| Tate) | |
| Microcare PE (Phenoxyethanol) | 0.89 |
| Microcare emollient PTG | 2.22 |
| (Pentylene Glycol) | |
| Total | 100 |

*Sufficient Quantity For

First, the preservatives were dispersed in the glycols (glycerol and propanediol) by stirring with a paddle stirrer. Finally, water was added, and the whole composition was mixed with a paddle stirrer for 1 hour.

The resulting aqueous phase is transparent and has a viscosity of less than 15 mPa·s.

### Example 2: Preparation of Capsules

The table below indicates the composition of simple capsules comprising a gelled alginate shell and a core of an oily phase.

| | | | Mass %/ | Mass %/ |
|---|---|---|---|---|
| Phase | Trade Name | INCI | phase | capsule |
| OF | Water | Water | 98.36 | 80.14 |
| | Protanal LF 200S | Sodium alginate | 1.5 | 1.22 |
| | SDS | Sodium lauryl | 0.14 | 0.11 |
| | | sulfate | | |
| IF | Sytenol A | Bakuchiol | 8 | 1.48 |
| | DUB MCT55/45 MB | Caprylic/Capric | 92 | 17.04 |
| | | triglyceride | | |

| | |
|---|---|
| Total | 100.00% |

### A. Preparation of the First Liquid Solution (IF)

Bakuchiol and oil were mixed, and stirring was maintained until homogenization.

### B. Preparation of the Second Liquid Solution (OF)

SDS and sodium alginate were added to water under magnetic stirring. The solution was kept under stirring for 24 hours to ensure that the alginate was completely dissolved and the solution was homogeneous.

### C. Preparation of the Gelling Solution

200 g of calcium chloride were dissolved in 1,000 g of water.

### D. Obtaining the Capsules

The capsule manufacturing process is based on the concentric co-extrusion of two solutions, notably described in FR2964017, implemented with the flow rates described in the table below. The capsules were thus obtained according to the following steps:
separate conveyance in a nozzle in the form of a double envelope of the first liquid solution (IF) and the second liquid solution (OF);
formation, at the exit of the double envelope, of a series of droplets, each droplet comprising a central core formed of the first solution (IF) and a peripheral film formed of the second solution (OF), completely covering the central core; the step of forming the series of droplets comprises a step of forming a liquid jet at the exit of the double envelope and a step of fragmenting the liquid jet into droplets;
immersion of each droplet in the gelling solution to change the sodium alginate of the second liquid solution (OF) from a liquid state to a gelled state and form the gelled shell, the central core forming the liquid core, thereby obtaining capsules; and
recovery of the formed capsules.

The flow rates are as follows:

| Phase | Flow rate per nozzle (in ml/hr) |
|---|---|
| OF | 40.74 |
| IF | 9.26 |

Simple macroscopic and monodisperse capsules with a size (or diameter) of 800 μm are obtained.

### Example 3: Preparation of Capsules

The table below indicates the composition of four types of simple capsules that differ from those described in Example 2 at the level of the gelled shell.

| | Trade | | A | B | C | D |
|---|---|---|---|---|---|---|
| Phase | Name | INCI | Mass %/phase | | | |
| OF | Water | Water | SQF* | | | |
| | Algogel | Sodium | 0.8 | 0.4 | 1 | 0 |
| | 1521 | alginate | | | | |
| | Kelcogel F | Gellan Gum | 0 | 0.4 | 0 | 0 |
| | Gellan | | | | | |
| | Gum | | | | | |
| | Pectin LM | Pectin | 0 | 0 | 0.5 | 0 |
| | VAHINE | Agar | 0 | 0 | 0 | 0.8 |
| | SDS | Sodium lauryl | 0 | 0.14 | 0.14 | 0.14 |
| | | sulfate | | | | |
| | Tween 80 | Polysorbate 80 | 1 | 0 | 0 | 0 |

| | Total | | 100 |
|---|---|---|---|
| IF | Sytenol A | Bakuchiol | 8 |
| | DUB | Caprylic/Capric | 92 |
| | MCT55/45 | triglyceride | |
| | MB | | |

| | |
|---|---|
| Total | 100.00% |

The manufacturing process and flow rates are identical to those described in Example 2. Test D differs in that (i) the OF is maintained at 80° C. and (ii) the gelling solution comprises only oil at 10° C.

Simple macroscopic and monodisperse capsules with a size (or diameter) of 800 μm are obtained.

### Example 4: Preparation of Pigmented Capsules

The table below indicates the composition of simple capsules that differ from those described in Example 2 at the level of the core.

| Phase | Trade Name | INCI | Mass %/phase |
|---|---|---|---|
| OF | Water | Water | SQF* |
| | Protanal LF 200S | Sodium alginate | 1.5 |
| | SDS | Sodium lauryl sulfate | 0.14 |
| | Total | | 100.00% |
| IF | KAK HL | Hexyl laurate | SQF* |
| | BENTONE GEL | OCTYLDODECANOL | 8.3 |
| | EUG V | AND | |
| | | DISTEARDIMONIUM | |
| | | HECTORITE | |
| | | AND PROPYLENE | |
| | | CARBONATE | |
| | ASL-1 TIO2 CR-50 | CI77891, Aluminum | 21.4 |
| | | hydroxide, Sodium | |
| | | Lauroyl glutamate, | |
| | | Lysine, Magnesium | |
| | | chloride | |
| | ASL-1 YELLOW LL- | CI 77492, Sodium | 2.8 |
| | 100P | Lauroyl glutamate, | |
| | | Lysine, Magnesium | |
| | | chloride | |
| | ASL-1 RED R-516P | CI 77491, Sodium | 0.7 |
| | | Lauroyl glutamate, | |
| | | Lysine, Magnesium | |
| | | chloride | |
| | ASL-1 BLACK BL- | CI 77499, Sodium | 0.4 |
| | 100P | Lauroyl glutamate, | |
| | | Lysine, Magnesium | |
| | | chloride | |

| | |
|---|---|
| Total | 100.00% |

*Sufficient Quantity For

The manufacturing process and flow rates are identical to those described in Example 2. Simple macroscopic and monodisperse capsules with a size (or diameter) of 800 μm are obtained.

### Example 5: Preparation of Capsules

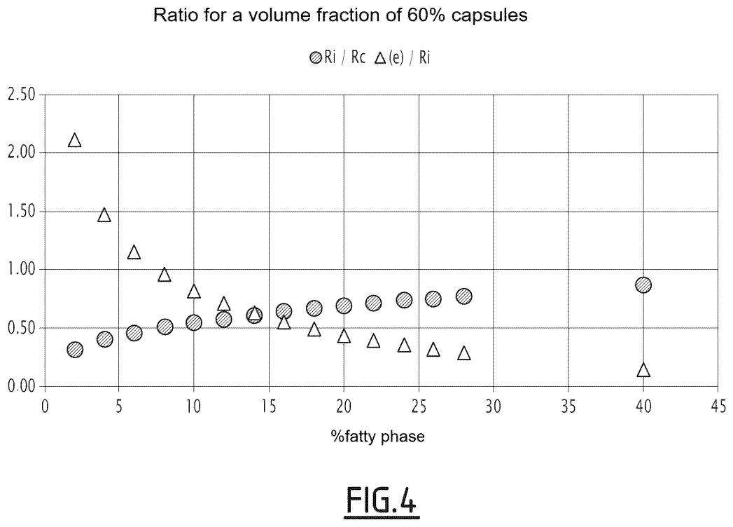

From the solutions and flow rates described below, perfuming capsules were manufactured using the microfluidic device illustrated in FIG. 4 of the patent application filed under no. FR2314864.

| | Raw | | |
|---|---|---|---|
| Solution | Materials | INCI | %* |
| Inverse | Water | Water | 50 |
| Emulsion | CaCl2 | Calcium chloride | 50 |
| (IF) | Total IF1 | | 100 |
| | PGPR | Polyglyceryl polyricinoleate | 0.05 |
| | Labrafac CC MB | Caprylic/Capric Triglyceride | SQF* |
| | Lipex 205 | Butyrospermum Parkii | 5 |
| | | (Shea) Butter | |
| | Vitamin E | Tocopherol | 0.8 |
| | Total IF2 | | 100 |
| | IF2 / IF1 Ratio | | 95 / 5 |
| Second | Water | Water | SQF* |
| Aqueous | Polyvinyl alcohol | Polyvinyl alcohol (PVA) | 1 |
| Phase | Total | 100 | 100 |
| (OF) | | | |
| Aqueous | Water | Water | SQF* |
| Solution | Sodium alginate | Sodium alginate | 0.3 |
| for | Ethanol | Ethyl alcohol | 20 |
| Shell | Total | | 100 |
| Formation | | | |
| (AF) | | | |

*Sufficient Quantity For.

| | | Flow Rates |
|---|---|---|
| | | (in ml/hr/nozzle) |
| Inverse Emulsion | Dispersed Aqueous Phase (IF1) | 0.7 |
| (IF) | Continuous Oily Phase (IF2) | 13 |

| | |
|---|---|
| Second Aqueous Phase (OF) | 1,000 |
| Aqueous Solution for Shell Formation (AF) | 700 |

Simple macroscopic and monodisperse capsules with a size (or diameter) of 1,200 μm are obtained.

### Example 6: Influence of the Volume Fraction

Example 6 involves contacting and mixing the capsules from Example 2 with the continuous aqueous phase from Example 1 in a 100 ml packaging equipped with a spray-type distribution pump, in different “continuous aqueous phase/capsules” ratios as described below. Note that the capsules were previously filtered and washed with osmosis water (to remove the gelling solution) before being mixed with the continuous aqueous phase from Example 1.

### Rating Criteria:

| | 0 | 1 | 2 |
|---|---|---|---|
| Suspensivity | No suspension; strong | Partial suspension; | Satisfactory |
| | creaming in less than 1 | slight creaming after 1 | suspension; |
| | min. at RT* | hour at RT* | no creaming after 1 |
| | | | month at RT* |
| Sprayability | Poor sprayability | Average sprayability | Good sprayability with |
| | and/or delivery | and/or inhomogeneous | homogeneous delivery |
| | | delivery | |
| Shine | Composition that does | Composition that | Composition that |
| | not reflect light | reflects light without | reflects light and |
| | | shining | shines |

*RT: Room Temperature

### Tests and Results

| | φ | φ | | Spray- | | Bakuchiol |
|---|---|---|---|---|---|---|
| Tests | PAC(1) | capsules(2) | Suspensivity | ability | Shine | rate |
| 3.1 | 70 | 30 | 0 | NA(3) | 0 | 0.45 |
| 3.2 | 60 | 40 | 1 | NA(3) | 0 | 0.59 |
| 3.3 | 50 | 50 | 2 | 1 | 1 | 0.74 |
| 3.4 | 40 | 60 | 2 | 2 | 2 | 0.89 |
| 3.5 | 30 | 70 | 2 | 1 | 2 | 1.04 |
| 3.6 | 20 | 80 | 2 | 0 | 2 | 1.19 |

(1)φ PAC: volume fraction of the Continuous Aqueous Phase according to Example 1 relative to the total volume of the composition.
(2)φ Capsules: volume fraction of the capsules according to Example 2 relative to the total volume of the composition.
(3)NA: Not Applicable

From the results above, it is observed that a volume fraction of capsules less than 50% does not allow for satisfactory steric suspension due to too rapid creaming of the capsules. In use, a preferential distribution of the continuous aqueous phase is observed, leading to an increase in capsule concentration as the product is used. Mechanically, the formulation of the distributed composition is inhomogeneous, i.e., mostly composed of the continuous aqueous phase at the beginning of use and then increasingly concentrated in the dispersed phase as use progresses. An exact and constant formulation of the product delivered throughout its use is therefore impossible to guarantee. This increase in capsule concentration even leads, after a while, to clogging of the aspiration/delivery device. This explains the “NA” rating for tests 3.1 and 3.2.

From a volume fraction of capsules of 50%, a composition capable of ensuring the suspension of the capsules in the continuous aqueous phase is achieved. Consequently, throughout the use of the product, an exact and constant formulation of the product delivered is observed, as well as the absence of obstruction or clogging at the aspiration/delivery device, which is particularly unexpected given the high concentration of capsules.

Finally, from a volume fraction of capsules of 70%, the product becomes less and less fluid to be homogeneously pumped. A significant degradation of the sprayable character of the composition occurs from a volume fraction of capsules of 80%.

Please note that all samples have very interesting aesthetic properties in the packaging. Indeed, in these samples, the macroscopic capsules, and in particular the core of the capsules, are perfectly distinguishable, which is all the easier as the continuous aqueous phase and the shell are transparent. Furthermore, it is noted that the volume fraction, in addition to the suspension of the capsules and sprayability, also impacts the more or less shiny character of the composition.

Finally, it is observed that tests 3.3 to 3.5 relate to compositions that offer great sprayability while maintaining the stability and properties of the active agent.

Similar observations were made with the capsules according to Examples 3 and 4 above, where the capsules were previously filtered and washed with osmosis water (to remove the OF, AF, and/or gelling solution) before being mixed with the continuous aqueous phase from Example 1.

### Example 7: Influence of the Ri/Rc Ratio

Example 7 involves contacting and mixing, in the continuous aqueous phase described in Example 1, capsules in a volume fraction of 60% relative to the total volume of the composition. These capsules differ from Example 2 in terms of flow rates, as indicated in the table below.

The obtained compositions are packaged in a 100 ml packaging equipped with a spray-type distribution pump.

FIG. 4 illustrates the evolution of the Ri/Rc ratio and the (e)/Ri ratio for these Tests 4.1 to 4.6.

| | Tests | | | | | |
|---|---|---|---|---|---|---|
| | 4.1 | 4.2 | 4.3 | 4.4 | 4.5 | 4.6 |
| | Comp. | Inv. | Inv. | Inv. | Inv. | Comp. |
| Flow rate OF⁽⁴⁾ | 46.67 | 43.33 | 38.33 | 32.5 | 25 | 13.11 |
| Flow rate IF⁽⁴⁾ | 3.33 | 6.67 | 11.67 | 17.5 | 25 | 36.89 |
| Ri (in microns) | 162 | 204 | 246 | 282 | 317 | 361 |
| (e) (in microns) | 238 | 196 | 154 | 118 | 83 | 39 |
| Rc (in microns) | 400 | 400 | 400 | 400 | 400 | 400 |
| Size (in | 800 | 800 | 800 | 800 | 800 | 800 |
| microns) | | | | | | |
| % oily phase/ | 4 | 8 | 14 | 21 | 30 | 45 |
| composition | | | | | | |
| Ri/Rc ratio⁽⁵⁾ | 0.41 | 0.51 | 0.62 | 0.71 | 0.79 | 0.90 |
| (e)/Ri ratio⁽⁵⁾ | 1.47 | 0.96 | 0.62 | 0.42 | 0.26 | 0.11 |

⁽⁴⁾Flow rate per nozzle (in ml/hr)
⁽⁵⁾Ri corresponds to the radius of the capsule core; Rc corresponds to the radius of the capsule (i.e., resulting from the sum of the capsule core radius and the shell thickness); (e) corresponds to the shell thickness.

### Rating Criteria:

| | 0 | 1 | 2 |
|---|---|---|---|
| Mechanical | Low Rm | Average Rm | Good Rm |
| Resistance | Capsule rupture in | Oily phase leakage to the | Stable capsules/composition, |
| (Rm)⁽⁶⁾ | packaging −> | continuous aqueous | no opacification |
| | opacification −> | phase −> shriveled or | |
| | strong visual | empty capsules, with | |
| | alteration and | opacification −> visual | |
| | creaming | alteration | |
| Sprayability | Poor sprayability, | Average sprayability | Good sprayability with |
| | even clogging | and/or inhomogeneous | homogeneous delivery |
| | (capsules too robust) | delivery | |
| Sensory | Very uncomfortable | Uncomfortable | Comfortable application with |
| quality⁽⁷⁾ | application; hard and | application; hard residual | few or no residual membranes |
| | persistent residual | membranes difficult to | that disappear easily and |
| | membranes upon | disappear upon | quickly upon application |
| | application | application | |

⁽⁶⁾Rm = Mechanical resistance, also referred to as “compression resistance.” The principle is to apply a constant speed constraint on a capsule and measure the force required to rupture the capsule. A method for measuring this mechanical resistance is notably described in WO2015075074.
⁽⁷⁾Sensory quality is notably reflected by easy and comfortable application of the composition on the material to be treated, and notably by the absence of residual membrane, at least the ability of these residual membranes, when present, to (i) not overly impact the visual of the composition applied on the material to be treated and (ii) to spread and disappear easily upon spreading.

### Tests and Results

| Tests | Mechanical Resistance | Sprayability | Sensory quality |
|---|---|---|---|
| 4.1 | 2 | 0 | 0 |
| 4.2 | 2 | 1 | 1 |
| 4.3 | 2 | 2 | 2 |
| 4.4 | 2 | 2 | 2 |
| 4.5 | 1 | 2 | 2 |
| 4.6 | 0 | 2 | 2 |

Test 4.1 is characterized by capsules with a very thick shell. Such capsules allow access to a very stable composition but with unsatisfactory sprayability and sensory quality.

Test 4.6 is characterized by capsules with a very large oily phase core. Such capsules allow access to a composition very loaded with active agent, but their robustness is insufficient.

Interesting results are obtained with Tests 4.2 to 4.5, for which the Ri/Rc ratio is between 0.5 and 0.8. The best results are obtained with Tests 4.3 and 4.5, and even 4.3 and 4.4, corresponding to an Ri/Rc ratio between 0.6 and 0.8, and particularly between 0.6 and 0.7. These Ri/Rc ratio values therefore correspond to the best non-obvious compromises between mechanical resistance, sprayability, and sensory quality.

Finally, it is observed that Tests 4.2 to 4.5 relate to compositions that offer great sprayability while maintaining the stability and properties of the active agent.

### Example 8: Composition According to the Invention with a Double Population of Capsules

Example 8 involves contacting and mixing, in a continuous aqueous phase according to Example 1, capsules according to Tests 4.3 and 4.5 above and according to the volume fractions described in the table below.

The capsules, however, differ from Tests 4.3 and 4.5 by the addition, in the oily phase, of coloring agents, namely a red dye (i.e., D&C Red no. 17 K7007 (INCI: CI 26100-0.001%) for the capsules according to Test 4.3 and a blue dye (i.e., Phat Blue DC 6204 (INCI: CI 61565/CI 60725-0.001%) for the capsules according to Test 4.5.

| | Volume fraction (in %) |
|---|---|
| Capsules according to Test 4.3 | 25 |
| Capsules according to Test 4.5 | 30 |
| Aqueous phase according to Example 1 | 45 |
| Total | 100 |

The obtained composition is packaged in a 100 ml packaging equipped with a spray-type distribution pump.

Thus, a cosmetic product comprising a volume fraction of capsules greater than 50% is obtained, and therefore capable of sterically suspending the capsules.

The double population of capsules, identical in size but of different densities, gives the product an unexpected visual/aesthetic effect.

Thus, under agitation, the composition appears as a dispersion wherein the red capsules and the blue capsules are uniformly mixed, giving the overall impression of a purple product (i.e., State 1).

At rest, a reorganization of the capsules is observed, resulting in creaming of the less dense 4.5 capsules and sedimentation of the denser 4.3 capsules (i.e., State 2). This color evolution of the composition according to the invention is all the more unexpected as it occurs without altering the suspension of the capsules in the continuous aqueous phase. After a certain time, the composition presents a color gradient between red capsules at the lower level and blue capsules at the upper level.

New agitation leads to a return to a composition in the form of State 1 described above.

What is claimed is:

1. A composition, in the form of a dispersion comprising a dispersed phase in the form of capsules and a continuous aqueous phase, wherein:
the capsules represent a volume fraction between 50% and 70% relative to the total volume of the composition;
the capsules comprise a shell and a core comprising at least one oily phase comprising at least one active agent; and
the capsules comprise an Ri/Rc ratio between 0.5 and 0.8, wherein:
Ri corresponds to the radius of the core; and
Rc corresponds to the radius of the capsule.

2. The composition according to claim 1, wherein the capsules have a diameter greater than or equal to 250 μm.

3. The composition according to claim 1, wherein the capsules are monodisperse.

4. The composition according to claim 1, wherein the volume fraction of the capsules is between 55% and 65% relative to the total volume of the composition.

5. The composition according to claim 1, wherein the shell is:
a gelled envelope completely encapsulating the core, said gelled envelope comprising at least one polyelectrolyte in the gelled state and/or at least one thermosensitive hydrophilic gelling agent; and/or
an envelope derived from a complex coacervation reaction between two oppositely charged polymers capable of coacervating.

6. The composition according to claim 5, wherein the composition comprises between 0.25% and 5% by weight of polyelectrolyte(s) and/or thermosensitive hydrophilic gelling agent(s) and/or oppositely charged polymers capable of coacervating, relative to the total weight of the shell.

7. The composition according to claim 1, wherein the shell further comprises at least one surfactant.

8. The composition according to claim 1, wherein the capsules comprise a liquid core or at least partially gelled or at least partially thixotropic, the core being monophase or including an intermediate oily phase droplet, and at least one internal droplet of an internal phase disposed in the intermediate droplet.

9. The composition according to claim 1, wherein the composition comprises between 5% and 40% by weight of oily phase relative to the total weight of the composition.

10. The composition according to claim 1, wherein the active agent is chosen from a cosmetic active agent, a biologically active product, an edible active agent, and their mixtures.

11. The composition according to claim 1, wherein the oily phase comprises from 0.01% to 30% by weight of active agent(s) relative to the total weight of the composition.

12. The composition according to claim 1, wherein the composition comprises from 0.1% to 100% by weight of active agent(s) relative to the total weight of the oily phase.

13. The composition according to claim 1, wherein the oily phase further comprises at least one oil.

14. The composition according to claim 1, wherein the continuous aqueous phase does not comprise carbomer.

15. The composition according to claim 1, wherein the continuous aqueous phase is liquid and is non-suspensive with respect to the capsules.

16. The composition according to claim 1, wherein the continuous aqueous phase has a high shear viscosity, as measured at 25° C. and under a shear stress of 100 s⁻¹, less than or equal to 100 mPa·s.

17. The composition according to claim 1, wherein the composition is sprayable.

18. A non-therapeutic cosmetic treatment process of keratinous material comprising at least one step of applying to a keratinous material at least one composition according to claim 1.

19. A method for the care and/or makeup of keratinous material comprising at least one step of applying to a keratinous material at least one composition according to claim 1.
